(19) United States
(12) Patent Application Publication
(10) Pub. No.: US 2018/0120309 A1
(43) Pub. Date: May 3, 2018

(54) ELISA PLATE

(71) Applicant: The First Affiliated Hospital of Jinzhou Medical University, Liaoning (CN)

(72) Inventor: Shaoheng HE, Liaoning (CN)

(21) Appl. No.: 15/565,560

(22) PCT Filed: Jun. 20, 2016
(86) PCT No.: PCT/CN2016/086355

§ 371 Date: Oct. 10, 2017

(30) Foreign Application Priority Data
Mar. 24, 2016 (CN) 201610173244.9

(51) Int. Cl. G01N 33/543

(52) U.S. Cl. CPC G01N 33/54386; G01N 33/54393

(57) ABSTRACT

An ELISA plate is provided, and includes several ELISA plate wells, each of which includes a hemispherical bottom, a funnel-shaped formation arranged in the middle and connected to the hemispherical bottom, and a projecting wall arranged in an upper part and connected to the funnel-shaped formation. The hemispherical bottom of the ELISA plate can increase the surface area of the ELISA plate over conventional designs, thereby increasing the opportunity of contact between a target substance and a target molecule on the surface of the solid phase and enabling detection of the content of the target substance using less sample amount. The funnel-shaped formation of the ELISA plate well is arranged to be connected to the bottom and upper part of the ELISA plate well, thereby further increasing the surface area of the ELISA plate well.

### CROSS-REFERENCE TO RELATED APPLICATIONS

This application is a national phase of PCT/CN2016/086355, filed on Jun. 20, 2016, which claims priority to Chinese Patent Application No. 201610173244.9, titled “ELISA PLATE”, and filed on Mar. 24, 2016, the entire contents of which are incorporated herein by reference.

### TECHNICAL FIELD

The present invention relates to the field of immunoassay devices, and in particular, to an ELISA plate.

### BACKGROUND

Immunoassay is a most commonly used method for detecting target molecules in the field of life science, and enzyme linked immunosorbent assay (ELISA) is an important part of immunoassay. ELISA mainly involves two reactions: one is an immunoreaction between an antigen and an antibody, and the other is a reaction between a biomolecule and a solid phase surface to which the biomolecule is adsorbed. An antigen and an antibody involved in the immunoreaction, a purity, a concentration and a proportion of a labeled antigen or antibody, and the conditions, such as the type of buffer, a concentration, an ion strength, a pH value, a reaction temperature, time, etc., play a key role in ELISA. In addition, the surface of an ELISA plate, as a carrier, also plays a very important role in adsorbing the antigen, the antibody or an antigen-antibody complex.

In the prior art, the most commonly used material for an ELISA plate is polystyrene. In recent years, in order to increase the affinity between a biomolecule and a solid phase surface, improve the reaction sensitivity, enhance the detection stability, etc., the selection and processing of a solid phase material of the ELISA plate have attracted great attention of researchers in this field. At present, most researchers in the field are dedicated to research on modifying the chemical property of the surface of polystyrene by activating a functional group using a covalently cross-linked chemical group, modifying the surface of polystyrene through chemical reaction, ultraviolet radiation, etc., thereby to improve the affinity of the ELISA plate well surface for biomolecules. Much progress has been made in this respect.

However, the polystyrene ELISA plate still has another problem, i.e. the problem with the surface area of the ELISA plate wells available to react with the biomolecules. In the prior art designs, the ELISA plate usually has a flat bottom (as shown in FIG. 1), a U-shaped bottom or a V-shaped bottom. The flat bottom has a low refractive index, and is applicable to ELISA detection. The ELISA plate having a U-shaped bottom has a high refractive index, is convenient for operations, such as sample addition, sample suction, full mixing, etc. The ELISA plate having a V-shaped bottom enables accurate sample suction. However, the surface area of the ELISA plate wells of the above ELISA plates available to react with the biomolecules is small, and can thus be unfavorable to make the reaction proceed sufficiently and fast.

### SUMMARY

In view of this, an object of the present invention is to provide an ELISA plate with a large surface area and a small sample loading amount. In order to realize the above object of the invention, the present invention provides the following technical solutions:

The present invention provides an ELISA plate, including several ELISA plate wells, each of which includes a hemispherical bottom, a funnel-shaped formation connected to an upper edge of the hemispherical bottom, and a projecting wall connected to an upper edge of the funnel-shaped formation.

Preferably, the hemispherical bottom includes a primary hemisphere and secondary hemispheres disposed inside the primary hemisphere and projecting inwardly.

Preferably, the primary hemisphere has a diameter of 0.2 to 10 mm, and a wall thickness of 0.1 to 1.0 mm.

Preferably, the number of the secondary hemispheres disposed inside the primary hemisphere is in a range of 3 to 50, and the secondary hemispheres are uniformly located on an inner surface of the primary hemisphere.

Preferably, each secondary hemisphere has a diameter of 0.1 to 5 mm, and a wall thickness of 0.1 to 1.0 mm, and a gap between adjacent secondary hemispheres is 0.1 to 1.0 mm.

Preferably, the funnel-shaped formation consists of a hollow spherical segment located in a lower part thereof and a truncated hollow cone connected to an upper edge of the hollow spherical segment, wherein a lower edge of the hollow spherical segment is connected to the upper edge of the hemispherical bottom; and an upper edge of the truncated hollow cone is connected to a lower edge of the projecting wall.

Preferably, the hollow spherical segment has a diameter of 0.2 to 10 mm, a height of 0.1 to 5.0 mm, and a wall thickness of 0.1 to 1.0 mm.

Preferably, the truncated hollow cone has a height of 0.2 to 6.0 mm, a wall thickness of 0.1 to 1.0 mm, a top radius of 0.1 to 5.0 mm, and a bottom radius of 0.1 to 5.0 mm.

Preferably, the projecting wall has a hollow cylindrical structure with a height of 0.1 to 20 mm, a wall thickness of 0.1 to 1.0 mm, and a radius of 0.1 to 5.0 mm.

Preferably, the ELISA plate has an area of 10 to 200 mm×5 to 200 mm, and each ELISA plate preferably includes 1 to 500 ELISA plate wells.

The ELISA plate provided by the present invention includes several ELISA plate wells, each of which includes a hemispherical bottom, a funnel-shaped formation connected to an upper edge of the hemispherical bottom and a projecting wall connected to an upper edge of the funnel-shaped formation. In the ELISA plate provided by the present invention, the hemispherical bottom can increase the surface area of the ELISA plate, thereby increasing the opportunity of contact between a target substance and a target molecule on the surface of the solid phase and enabling detection of the content of the target substance using less sample amount; the funnel-shaped formation is arranged to be connected to a bottom and an upper part of each ELISA plate well, thereby further increasing the surface area of the ELISA plate wells; and the projecting wall can prevent cross-contamination between the ELISA plate wells.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, explain the one or more embodiments of the invention:

FIG. 1 is a perspective view of an ELISA plate well according to one prior art design;

FIG. 2A shows a top view of an ELISA plate well in accordance with one embodiment of the present invention;

FIG. 2B shows a front view of the ELISA plate well of FIG. 2A;

FIG. 3 is a cross-sectional view of the ELISA plate well of FIG. 2A;

FIG. 4 is a perspective view of the ELISA plate well of FIG. 2A;

FIG. 5A shows a top view of a 20-well ELISA plate in accordance with another embodiment of the present invention;

FIG. 5B shows a side view of the 20-well ELISA plate of FIG. 5A;

FIG. 5C shows a top view of a 28-well ELISA plate in accordance with another embodiment of the present invention;

FIG. 5D shows a side view of the 28-well ELISA plate of FIG. 5C;

FIG. 6A shows a cross-sectional view of the 20-well ELISA plate of FIGS. 5A and 5B;

FIG. 6B shows a detailed cross-sectional view of one of the wells of the ELISA plates of FIGS. 6A and 6B;

FIG. 6C shows a cross-sectional view of the 28-well ELISA plate of FIGS. 5C and 5D;

FIG. 7A shows a perspective view of the ELISA plate of FIGS. 5A and 5B; and

FIG. 7B shows a perspective view of the ELISA plate of FIGS. 5C and 5D.

### DETAILED DESCRIPTION

The present invention is further illustrated below in conjunction with the embodiments and the accompanying drawings.

The present invention provides an ELISA plate, including several ELISA plate wells, each of which includes a hemispherical bottom, a funnel-shaped formation arranged in the middle and connected to the hemispherical bottom, and a projecting wall arranged in the upper part and connected to the funnel-shaped formation.

In the present invention, the ELISA plate has an area of 10 to 200 mm×5 to 200 mm. Each ELISA plate preferably includes 1 to 500 ELISA plate wells, more preferably 10 to 400 ELISA plate wells, and most preferably 16 or 40 ELISA plate wells. A density of the wells in the ELISA plate is preferably 0.2 well/cm2 to 8.0 wells/cm2, and more preferably 0.5 well/cm2 to 4 wells/cm2. The arrangement mode of the ELISA plate wells is not specifically defined herein, and any arrangement mode of the ELISA plate wells well known to those skilled in the art may be available. In the embodiments of the present invention, the ELISA plate wells are preferably arranged in a rectangular array, and a 20-well ELISA plate arranged in a 4×5 rectangular array or a 28-well ELISA plate arranged in a 4×7 rectangular array is more preferably used.

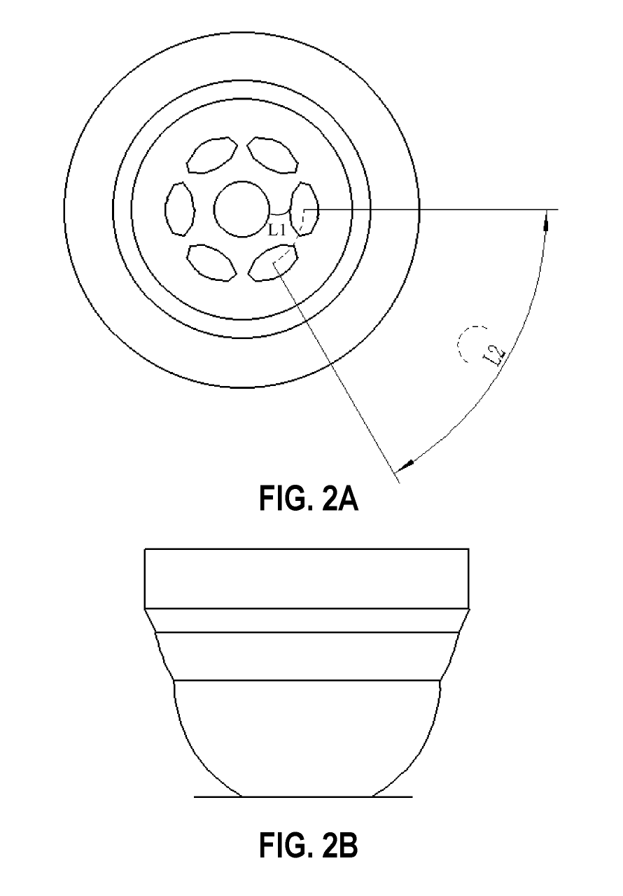

In the present invention, a structure of the ELISA plate well in one embodiment is shown in FIGS. 2A through 4. Each of the ELISA plate wells (1) includes a hemispherical bottom. The bottom of the ELISA plate well is configured to be a hemispherical structure so that the surface area of the ELISA plate can be increased, thereby increasing the opportunity of contact between a target substance and a target molecule on the surface of the solid phase and enabling detection of the content of the target substance using less sample amount. In the present invention, preferably, the hemispherical bottom includes a primary hemisphere (2) and secondary hemispheres (3) disposed inside the primary hemisphere and projecting inwardly. In the present invention, the primary hemisphere preferably has a diameter of 0.2 to 10 mm, more preferably 2 to 8 mm, and most preferably 6 to 7 mm.

According to the present invention, preferably, secondary hemispheres projecting inwardly are disposed inside each primary hemisphere. The number of the secondary hemispheres inside each primary hemisphere is 3 to 50, more preferably 5 to 15, and most preferably 7 to 9. In the present invention, the secondary hemispheres are uniformly located on an inner surface of the primary hemisphere.

In the present invention, the diameter of each secondary hemisphere is preferably 0.1 to 5 mm, more preferably 0.5 to 3 mm, and most preferably 1 to 2 mm. In the present invention, an arc length between adjacent secondary hemispheres is preferably 0.1 to 2.0 mm. In the present invention, a gap between adjacent secondary hemispheres is preferably 0.1 to 1.0 mm, more preferably 0.4 to 0.6 mm, and most preferably 0.5 mm. In the present invention, each gap between adjacent secondary hemispheres creates a liquid flow path of the ELISA plate well, so as to allow liquid to freely flow inside the ELISA plate wells and thus increase the opportunity of contact between a target substance and a target molecule of the solid phase.

In the present invention, each of the ELISA plate wells includes a funnel-shaped formation (4) connected to an upper edge of the hemispherical bottom. The funnel-shaped formation is arranged in a middle part of the ELISA plate well, and is connected upwardly to a lower edge of the projecting wall (7). In the present invention, the funnel-shaped formation consists of a hollow spherical segment (5) disposed in a lower part of the funnel-shaped formation and a truncated hollow cone (6) disposed in an upper part of the funnel-shaped formation. A lower edge of the hollow spherical segment is connected to the upper edge of the hemispherical bottom at the bottom of the ELISA plate well. An upper edge of the truncated hollow cone is connected to the lower edge of the projecting wall.

In the present invention, the diameter of the hollow spherical segment is preferably 0.2 to 10 mm, more preferably 3 to 8 mm, and most preferably 4.8 mm. The height of the hollow spherical segment is preferably 0.1 to 5.0 mm, more preferably 0.5 to 3 mm, and most preferably 0.8 mm. The wall thickness of the hollow spherical segment is preferably 0.1 to 1.0 mm, more preferably 0.3 to 0.8 mm, and most preferably 0.7 mm.

In the present invention, the height of the truncated hollow cone disposed in the upper part of the funnel-shaped formation is preferably 0.2 to 6.0 mm, more preferably 0.3 to 3 mm, and most preferably 0.4 mm. The wall thickness of the truncated hollow cone is preferably 0.1 to 1.0 mm, more preferably 0.2 to 0.8 mm, and most preferably 0.7 mm. The top radius of the truncated hollow cone is preferably 0.1 to 5.0 mm, and more preferably 1 to 4 mm. The bottom radius of the truncated hollow cone is preferably 0.1 to 5 mm, and more preferably 1 to 4 mm.

In the present invention, the projecting wall arranged in the upper part and connected to the upper edge of the funnel-shaped formation is preferably a hollow cylindrical structure. The height of the projecting wall is preferably 0.1 to 20 mm, more preferably 0.5 to 5.0 mm, and most preferably 1.0 mm. The wall thickness of the projecting wall is preferably 0.1 to 1.0 mm, more preferably 0.3 to 0.8 mm, and most preferably 0.7 mm. The radius of the hollow cylindrical structure of the projecting wall is preferably 0.1 to 5.0 mm, and more preferably 1.0 to 4.0 mm. In the present invention, the projecting wall plays a role in preventing cross contamination between the ELISA plate wells during the experiment.

In the present invention, an inner wall of the hemispherical bottom of each ELISA plate well is smoothly connected to an inner wall of the funnel-shaped formation, and an inner wall of the upper edge of the funnel-shaped formation is smoothly connected to an inner wall of the projecting wall.

The ELISA plate provided by the present invention is illustrated in detail below in conjunction with specific exemplary embodiments, which, however, shall not be construed as limiting the protection scope of the present invention.

### Embodiment 1

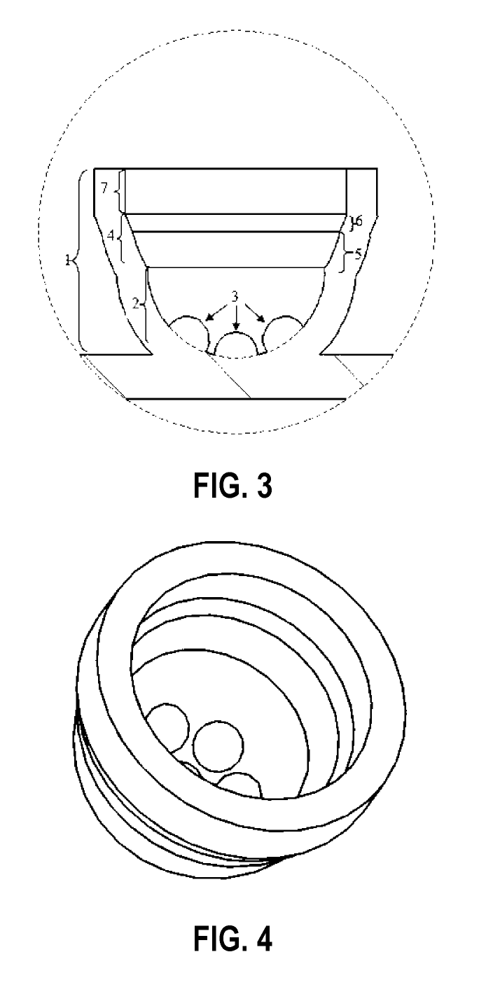

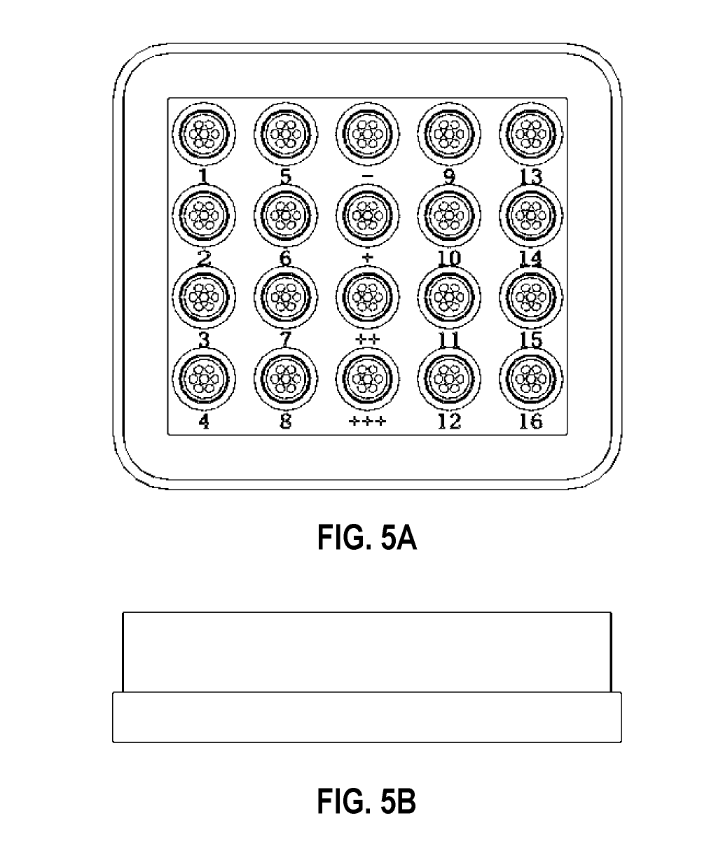

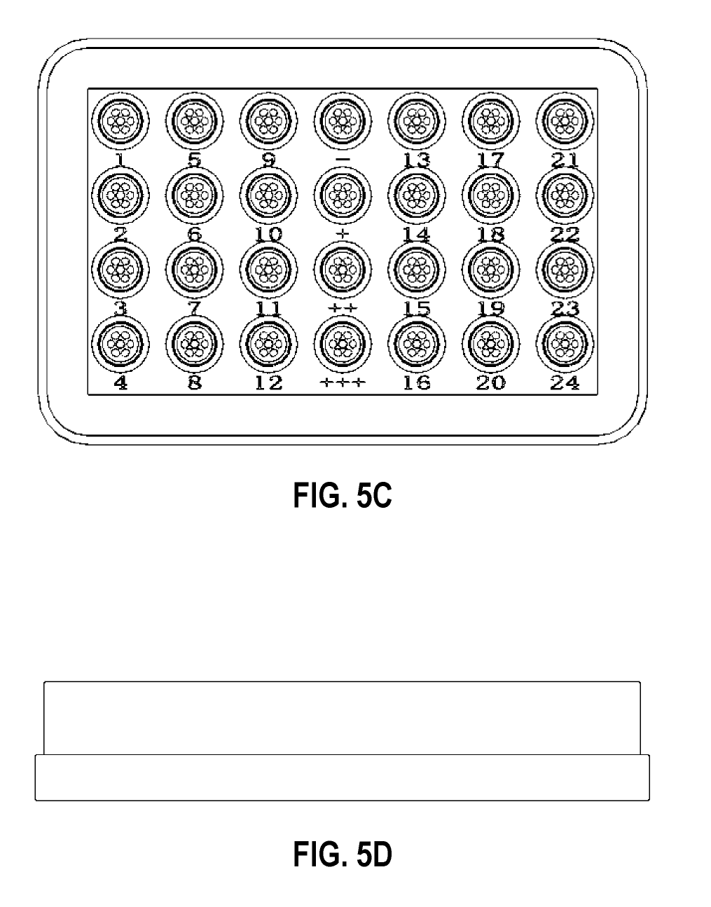

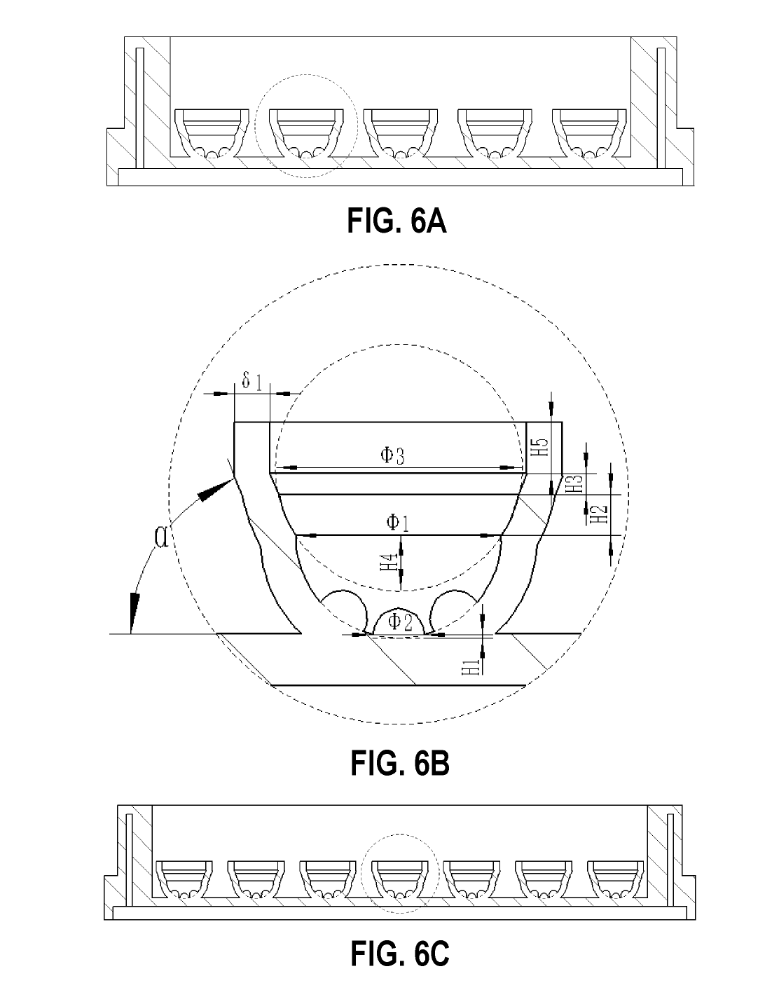

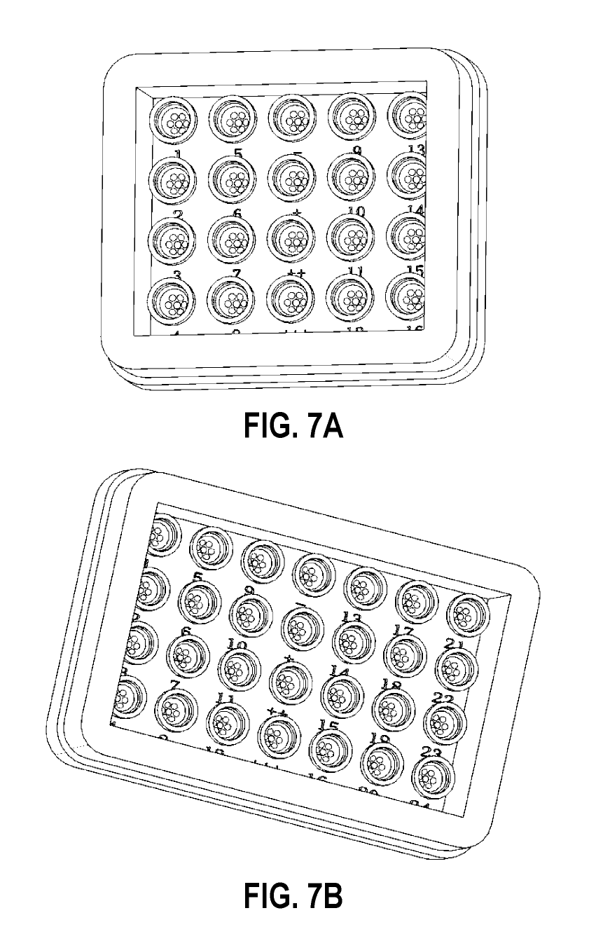

A cross sectional view of an ELISA plate in this embodiment is shown in FIG. 6A, a perspective view thereof is shown in FIG. 7A, a side view thereof is shown in FIG. 5B, and wells therein are arranged as shown in FIG. 5A. The ELISA plate includes 20 ELISA plate wells arranged in a 4×5 rectangular array. A top view and a front view of the ELISA plate well are shown in FIGS. 2A and 2B, respectively, and a cross sectional view thereof is shown in FIG. 3. In these figures, 1 denotes an ELISA plate well, 2 denotes a primary hemisphere, 3 denotes a secondary hemisphere, 4 denotes a funnel-shaped formation, 5 denotes a spherical segment in a lower part of the funnel-shaped formation, 6 denotes a truncated hollow cone in an upper part of the funnel-shaped formation, and 7 denotes a projecting wall. A perspective view of the structure of the ELISA plate well is shown in FIG. 4. Each of the ELISA plate wells includes a hemispherical bottom, a funnel-shaped formation arranged in a middle part and connected to the hemispherical bottom, and a projecting wall arranged in an upper part and connected to the funnel-shaped formation.

A cross sectional view of the ELISA plate wells is shown in FIGS. 6A and 6B, in which Φ1 denotes a diameter of the primary hemisphere at the bottom of the ELISA plate well, Φ2 denotes a diameter of the secondary hemisphere, Φ3 denotes a diameter of the hollow spherical segment in the lower part of the funnel-shaped formation in the middle part of the ELISA plate well, H1 denotes a shortest distance from the sphere center of the secondary hemisphere to the bottom surface of the primary hemisphere, H2 denotes a height of the hollow spherical segment in the lower part of the funnel-shaped formation in the middle part of the ELISA plate well, H3 denotes a height of the truncated hollow cone in the upper part of the funnel-shaped formation, and H4 denotes a shortest distance from a lower section of the hollow spherical segment in the lower part of the funnel-shaped formation to the sphere.

Specifically, the hemispherical bottom of each ELISA plate well is provided with a primary hemisphere and secondary hemispheres. The diameter Φ1 of the primary hemisphere is 4.0 mm. There are 7 secondary hemispheres arranged at the bottom of the primary hemisphere. The diameter Φ2 of each secondary hemisphere is 1.0 mm. The shortest distance H1 from the sphere center of the secondary hemisphere to the bottom surface of the primary hemisphere is 0.06 mm. Arc lengths L1 and L2 between adjacent secondary hemispheres are 0.21 mm and 1.16 mm, respectively.

A hollow spherical segment having a diameter Φ3=4.7738 mm, a height H2=0.8 mm and a wall thickness δ=0.7 mm is arranged in the lower part of the funnel-shaped formation in the middle part of each ELISA plate well. The shortest distance H4 from the lower section of the hollow spherical segment to the sphere is 1.01482 mm. The height H3 of the truncated hollow cone in the upper part of the funnel-shaped formation is 0.4 mm, and its wall thickness δ1 is 0.7 mm.

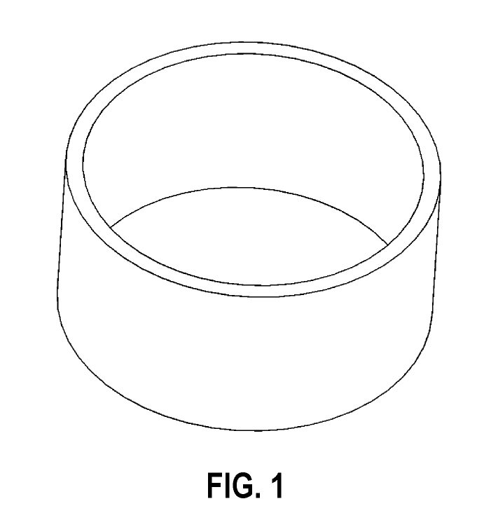

The height H5 of the projecting wall in the upper part of the ELISA plate well is 1.0 mm, and its wall thickness M is 0.7 mm. A comparison between the surface area inside the ELISA plate well as shown in FIGS. 6A and 6B and the surface area of a flat-bottomed ELISA plate well in the prior art (as shown in FIG. 1) may be calculated as follows:
surface area of the prior art flat-bottomed ELISA plate well:

$${S}_{Flat \text{-} bottomed ELISA plate well} = π \times {( \frac{{Φ}_{1}}{2} )}^{2} = 4 π ( {mm}^{2} ) ;$$

surface area of the primary hemisphere at the bottom of the ELISA plate well in this embodiment:

$${S}_{Primary hemisphere} = π \frac{{Φ}_{1}^{2}}{2} = 8 π ( {mm}^{2} ) ;$$

surface area of the secondary hemispheres at the bottom of the ELISA plate well in this embodiment:

$${S}_{Secondary hempheres} = 7 π \frac{{Φ}_{2}^{2}}{2} = 3.5 π ( {mm}^{2} ) ;$$

surface area of the spherical segment in the lower part of the funnel-shaped formation in the middle part of the ELISA plate well in this embodiment:

S_{Spherical segment}=πΦ₃H₂=3.81904π(mm²);
surface area of the primary hemisphere covered by the secondary hemispheres in this embodiment

S_{Primary hemisphere covered}=7πΦ₁H₁=1.68π(mm²);
total surface area of the ELISA plate well in this embodiment:

S_{Total}=S_{Primary hemisphere}+S_{Secondary hemispheres}+S_{Spherical segment}−S_{primary hemisphere covered}=8π+3.5π+3.81904π−1.68π=13.639π(mm²); and

ratio of the total surface area S_{Total} of the ELISA plate well in this embodiment to the surface area S of the prior art well:

$$\frac{{S}_{Total}}{{S}_{Flat \text{-} bottomed ELISA plate well}} = \frac{13.63904 π}{4 π} = 3.410 .$$

As can be seen from the above results, the total surface area of the ELISA plate well according to the present invention is 3.410 times as much as that of the prior art ELISA plate well. Accordingly, in accordance with the present invention, the surface area inside the ELISA plate well is increased significantly.

### Embodiment 2

Another embodiment of an ELISA plate is provided. A cross sectional view of the ELISA plate is shown in FIG. 6C, a perspective view thereof is shown in FIG. 7B, a side view of the ELISA plate is shown in FIG. 5D, and wells in the ELISA plate are arranged as shown in FIG. 5C. The ELISA plate includes 28 ELISA plate wells arranged in a 4×7 rectangular array. A top view and a front view of the ELISA plate well are shown in FIGS. 2A and 2B, respectively, and a cross sectional view thereof is shown in FIG. 3. In these figures, 1 denotes an ELISA plate well, 2 denotes a primary hemisphere, 3 denotes a secondary hemisphere, 4 denotes a funnel-shaped formation, 5 denotes a spherical segment in a lower part of the funnel-shaped formation, 6 denotes a truncated hollow cone in an upper part of the funnel-shaped formation, and 7 denotes a projecting wall. A perspective view of the ELISA plate well is shown in FIG. 4. Each of the ELISA plate wells includes a hemispherical bottom, a funnel-shaped formation arranged in a middle part and connected to the hemispherical bottom, and a projecting wall arranged in an upper part and connected to the funnel-shaped formation.

A cross sectional view of the ELISA plate wells is shown in FIGS. 6B and 6C, in which Φ1 denotes a diameter of the primary hemisphere at the bottom of the ELISA plate well, Φ2 denotes a diameter of the secondary hemisphere, Φ3 denotes a diameter of the hollow spherical segment in the lower part of the funnel-shaped formation in the middle part of the ELISA plate well, H1 denotes a shortest distance from the sphere center of the secondary hemisphere to the bottom surface of the primary hemisphere, H2 denotes a height of the hollow spherical segment in the lower part of the funnel-shaped formation in the middle part of the ELISA plate well, H3 denotes a height of the truncated hollow cone in the upper part of the funnel-shaped formation, and H4 denotes a shortest distance from a lower section of the hollow spherical segment in the lower part of the funnel-shaped formation to the sphere.

Specifically, the hemispherical bottom of each ELISA plate well is provided with a primary hemisphere and secondary hemispheres. The diameter 11 of the primary hemisphere is 4.0 mm. There are 7 secondary hemispheres arranged at the bottom of the primary hemisphere. The diameter Φ2 of each secondary hemisphere is 1.0 mm. The shortest distance H1 from the sphere center of the secondary hemisphere to the bottom surface of the primary hemisphere is 0.06 mm. Arc lengths L1 and L2 between adjacent secondary hemispheres are 0.21 mm and 1.16 mm, respectively.

A hollow spherical segment having a diameter Φ3=4.7738, a height H2=0.8 mm and a wall thickness δ1=0.7 mm is arranged in the lower part of the funnel-shaped formation in the middle part of each ELISA plate well. The shortest distance H4 from the lower section of the hollow spherical segment to the sphere is 1.01482 mm. The height H3 of the truncated hollow cone in the upper part of the funnel-shaped formation is 0.4 mm, and its wall thickness δ1 is 0.7 mm. The height H5 of the projecting wall in the upper part of the ELISA plate well is 1.0 mm, and its wall thickness is 0.7 mm.

A comparison between the surface area inside the ELISA plate well as shown in FIGS. 6B and 6C and the surface area of a prior art flat-bottomed ELISA plate well in the prior art is as follows:
surface area of the prior art flat-bottomed ELISA plate well:

$${S}_{Flat \text{-} bottomed ELISA plate well} = π \times {( \frac{{Φ}_{1}}{2} )}^{2} = 4 π ( {mm}^{2} ) ;$$

surface area of the primary hemisphere at the bottom of the ELISA plate well in this embodiment:

$${S}_{Secondary hempheres} = π \frac{{Φ}_{2}^{2}}{2} = 8 π ( {mm}^{2} ) ;$$

surface area of the secondary hemispheres at the bottom of the ELISA plate well in this embodiment:

$${S}_{Secondary hempheres} = 7 π \frac{{Φ}_{2}^{2}}{2} = 3.5 π ( {mm}^{2} ) ;$$

surface area of the spherical segment in the lower part of the funnel-shaped formation in the middle part of the ELISA plate well in this embodiment:

S_{Spherical segment}=πΦ₃H₂=3.81904π(mm²);
surface area of the primary hemisphere covered by the secondary hemispheres in this embodiment:

S_{Primary hemisphere covered}=7πΦ1H₁=1.68π(mm²);
total surface area of the ELISA plate well in this embodiment:

S_{Total}=S_{Primary hemisphere}+S_{Secondary hemispheres}+S_{Spherical segment}−S_{Primary hemisphere covered}=8π+3.5π+3.81904π−1.68π=13.639π(mm²); and
ratio of the total surface area S_{Total} of the ELISA plate well in this embodiment to the surface area S of a prior art well:

$$\frac{{S}_{Total}}{{S}_{Flat \text{-} bottomed ELISA plate well}} = \frac{13.63904 π}{4 π} = 3.410 .$$

As can be seen from the above embodiments, the total surface area of the ELISA plate well according to the present invention is 3.410 times as much as that of the prior art ELISA plate well. Accordingly, in accordance with the present invention, the surface area inside the ELISA plate well is increased significantly, thereby increasing the opportunity of contact between a target substance and a target molecule on the surface of the solid phase and enabling detection of the content of the target substance using less sample amount. The funnel-shaped formation of the ELISA plate well according to the present invention is configured to be connected to the bottom and upper part of each ELISA plate well, thereby further increasing the surface area of the ELISA plate wells. The projecting wall according to the present invention can prevent cross-contamination between the ELISA plate wells.

The foregoing illustration of the embodiments is only to help in understanding the methodology and concept of the present invention. It should be noted that for those skilled in the art, various improvements and modifications of the present invention can be made without departing from the principle of the present invention, and such improvements and modifications also fall within the scope of the present invention as claimed. Multiple amendments to these embodiments are obvious to those skilled in the art, and general principles defined in this application can be achieved in the other embodiments in case of not breaking away from the spirit or scope of the present invention. Thus, the present invention will be not limited to these embodiments shown in this application, but shall accord with the widest scope consistent with the principles and novel characteristics disclosed by this application.

What is claimed is:

1. An ELISA plate, comprising:
a number of ELISA plate wells, each of which comprises a hemispherical bottom, a funnel-shaped formation connected to an upper edge of the hemispherical bottom, and a projecting wall connected to an upper edge of the funnel-shaped formation.

2. The ELISA plate according to claim 1, wherein the hemispherical bottom comprises a primary hemisphere and secondary hemispheres disposed inside the primary hemisphere and projecting inwardly.

3. The ELISA plate according to claim 2, wherein the primary hemisphere has a diameter of 0.2 to 10 mm, and a wall thickness of 0.1 to 1.0 mm.

4. The ELISA plate according to claim 2, wherein a number of the secondary hemispheres inside the primary hemisphere is in a range of 3 to 50, and the secondary hemispheres are uniformly located on an inner surface of the primary hemisphere.

5. The ELISA plate according to claim 2, wherein the secondary hemisphere has a diameter of 0.1 to 5 mm, and a wall thickness of 0.1 to 1.0 mm, and a gap between adjacent secondary hemispheres is 0.1 to 1.0 mm.

6. The ELISA plate according to claim 1, wherein the funnel-shaped formation consists of a hollow spherical segment in a lower part thereof and a truncated hollow cone connected to an upper edge of the hollow spherical segment; wherein, a lower edge of the hollow spherical segment is connected to the upper edge of the hemispherical bottom, and an upper edge of the truncated hollow cone is connected to a lower edge of the projecting wall.

7. The ELISA plate according to claim 6, wherein the hollow spherical segment has a diameter of 0.2 to 10 mm, a height of 0.1 to 5.0 mm, and a wall thickness of 0.1 to 1.0 mm.

8. The ELISA plate according to claim 6, wherein the truncated hollow cone has a height of 0.2 to 6.0 mm, a wall thickness of 0.1 to 1.0 mm, a top radius of 0.1 to 5.0 mm, and a bottom radius of 0.1 to 5.0 mm.

9. The ELISA plate according to claim 1, wherein the projecting wall has a hollow cylindrical structure with a height of 0.1 to 20 mm, a wall thickness of 0.1 to 1.0 mm, and a radius of 0.1 to 5.0 mm.

10. The ELISA plate according to claim 1, wherein the ELISA plate has an area of 10 to 200 mm×5 to 200 mm, and each ELISA plate preferably comprises 1 to 500 ELISA plate wells.
